(19) United States
(12) Patent Application Publication
(10) Pub. No.: US 2005/0203575 A1
(43) Pub. Date: Sep. 15, 2005

(54) Skin microactivation system and method

(75) Inventors: Robert George Carson, Rahway, NJ (US); Surajit Mukherjee, Ridgewood, NJ (US); Uma Santhanam, Tenafly, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/800,997

(22) Filed: Mar. 15, 2004

(51) Int. Cl. A61B 17/50

(52) U.S. Cl. 606/204.35; 606/131

(57) ABSTRACT

The present invention relates to a system and technique using same for microactivating an area of the epidermis. The system includes a device and a skin benefit agent. The device microactivates epidermal tissue with efficiency and minimal discomfort. The system and method may be provided as part of a home-use kit for individualized use.

### FIELD OF THE INVENTION

The present invention relates to a skin microactivation system and method. More particularly, the present invention relates to a microactivation system and method for painlessly microactivating skin using a system of one or more needles.

### BACKGROUND OF THE INVENTION

Needles are well known to be used to deliver a drug or medicine or to extract samples from living tissue in order to analyze the samples for diagnostic purposes. The majority of such needles have been macroneedles, having a relatively large diameter relative to a blood cell, i.e., on the order of a millimeter. The vivida-sa.co.za website describes a macroneedle device referred to as the ENVIRON Medical ROLL-CIT™. For some applications, it is advantageous to use smaller diameter needles, i.e., microneedles, with diameters on the order of micrometers. Microneedle devices, as well as patches including needle-like projections, for pharmaceutical delivery and monitoring physiological and metabolic parameters are disclosed in, for example, U.S. Pat. Nos. 5,879,326 and 5,591,139. Yuzhakov, et al., U.S. Pat. Nos. 6,565,532 and 6,652,478 relate to microneedle apparatus used for marking skin and for dispensing semi-permanent subcutaneous makeup.

A need exists for a system for microactivation of skin for a cosmetic benefit. Furthermore, there is a need for a microactivation system and technique that does not require the presence of a physician, is minimally invasive, and can be done with minimal discomfort.

### SUMMARY OF THE INVENTION

While prior microneedles may be used with the inventive methods, to attenuate the shortcomings of non-suitable devices and prior methods and techniques, a skin microactivation system and technique have been developed. The present invention includes a system and method for microactivating epidermal cells at appropriate depths below the skin surface without substantially disrupting underlying dermis layers, so as to activate skin repair and regeneration functions for ultimate cosmetic benefit.

In a first aspect, the present invention provides a microactivation system including:

(a) a device for microactivation of a skin surface of an individual, including:

an actuator device comprising a central shaft having a proximal end and a distal end conjoined with a handle device; said central shaft having a central opening therein extending from said proximal end to said distal end;

a probe capable of being disposed centrally and releasably within the central opening of the actuator device; the probe comprising a cylindrical rod having a distal end and terminating in a surface at a cross-sectional proximal end thereof; said surface having a small cross-sectional diameter and having at least one needle or a plurality of needles protruding/extending therefrom; and

(b) a skin benefit agent.

In a second aspect, the present invention provides a method for microactivation of epidermal cells of a skin surface of an individual, by applying to the skin surface the inventive microactivation system or derivatives thereof, thereby microactivating epidermal tissue. For example, a plurality of needles may be arranged as an array on a transdermal patch, such an adhesive patch or a roller, for example, which may be painlessly applied to the epidermal skin, without penetrating the dermis. Generally, skin penetration is to a depth of about 100 micro-m.

In another aspect, the present invention is a kit including the microactivation device and/or skin benefit agent in accordance with the first aspect. The kit is suitable for consumer use and does not require a physician's assistance.

In a further aspect, a method for assembling the microactivation device of the inventive system is provided, including the steps of:
(a) securing the collet to the driver rod,
(b) inserting the probe into the collet,
(c) inserting the secured driver rod in the central opening of the central shaft of the actuator device; and
(d) tightening the knurled disc over the actuator device, thereby securing the driver rod therein and forcing the collet to grip the probe.

For a more complete understanding of the above and other features and advantages of the invention, reference should be made to the following description of the preferred embodiment.

### BRIEF DESCRIPTION OF THE DRAWINGS

The invention will be better understood by way of the following detailed description of a preferred embodiment, with reference to the appended drawings in which:

FIG. 1 is a perspective view of an assembled microactivation device according to one preferred embodiment;

FIG. 1A is an exploded perspective view of the microactivation device of FIG. 1, with the positioning sleeve removed;

FIG. 1B is an exploded perspective view of the actuator device and probe of the microactivation device of FIG. 1;

FIG. 2 is a cross-sectional view taken on the line 2-2 in FIG. 1;

FIG. 3 is a cross-sectional view taken along line 3-3 in FIG. 1B;

FIG. 4 is an enlarged top view of the surface of a probe in the microactivation device of FIG. 1;

FIG. 5 is a graph showing the effect of microactivation on BCL-2 expression in cultured keratinocytes;

FIG. 6 is a graph showing the effect of microactivation on Keratin 16 expression in cultured keratinocytes;

FIG. 7 is a graph showing the expression of procollagen 1 in fibroblasts treated with wounded keratinocyte-conditioned medium;

FIG. 8 is a graph showing the effect on CRABP II in human epidermis of combined treatment using microactivation device of FIG. 1 and retinol; and

FIG. 9 is a graph showing the effect of microactivation using the inventive method and device of FIG. 1 on BCL-2 expression in human epidermis.

### DETAILED DESCRIPTION OF THE INVENTION

The present invention is based on a new idea that barrier disruption of the skin and microwounding of basal skin cells can lead to activation of skin cells and result in beneficial cosmetic effects to the skin, such as, without wishing to be bound by theory, the wound healing and/or cell replacement/regeneration process of skin cells. The skin microactivation system and method of the present invention involve the disruption of the upper skin layer by applying a probe having needle-like projections, thereby activating the basal layer epidermal cells. Using the inventive device and method, the epidermis, including the basal layer of the epidermis, is disrupted, thereby effectively activating the skin's wound healing and/or cell regeneration process.

As used herein, the term “comprising” means including, made up of, composed of, consisting and/or consisting essentially of. Except in the operating and comparative examples, or where otherwise explicitly indicated, all numbers in this description indicating amounts or ratios of material or conditions of reaction, physical properties of materials and/or use are to be understood as modified by the word “about”.

The term “epidermis” refers to the outer nonvascular layer of the skin that overlies the dermis.

The term “microactivation” as used herein refers to the activation of the wound healing and/or beneficial barrier repair and/or regeneration and/or replacement mechanism or process of epidermal cells using microscopic scale devices to effect microscopic scale mechanical skin disruption or breach of the skin barrier/stratum corneum and microwounding of basal layer keratinocytes.

The term “skin” as used herein includes the skin on or in the face, mouth (epithelial cells), neck, chest, back, arms, hands, legs, and scalp.

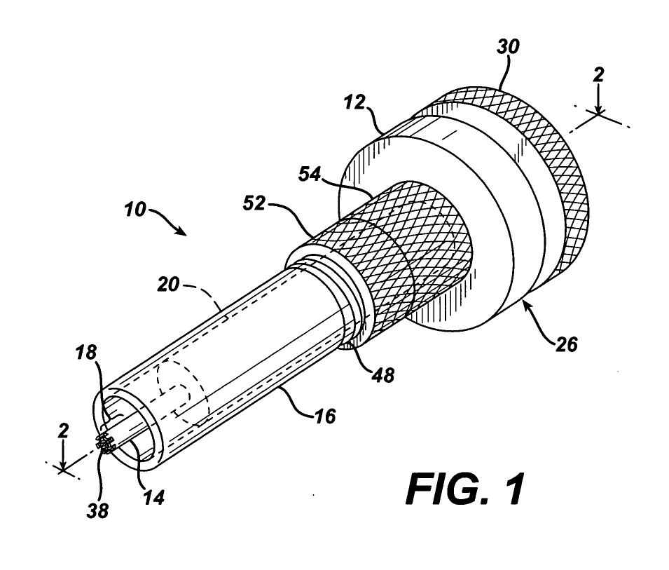

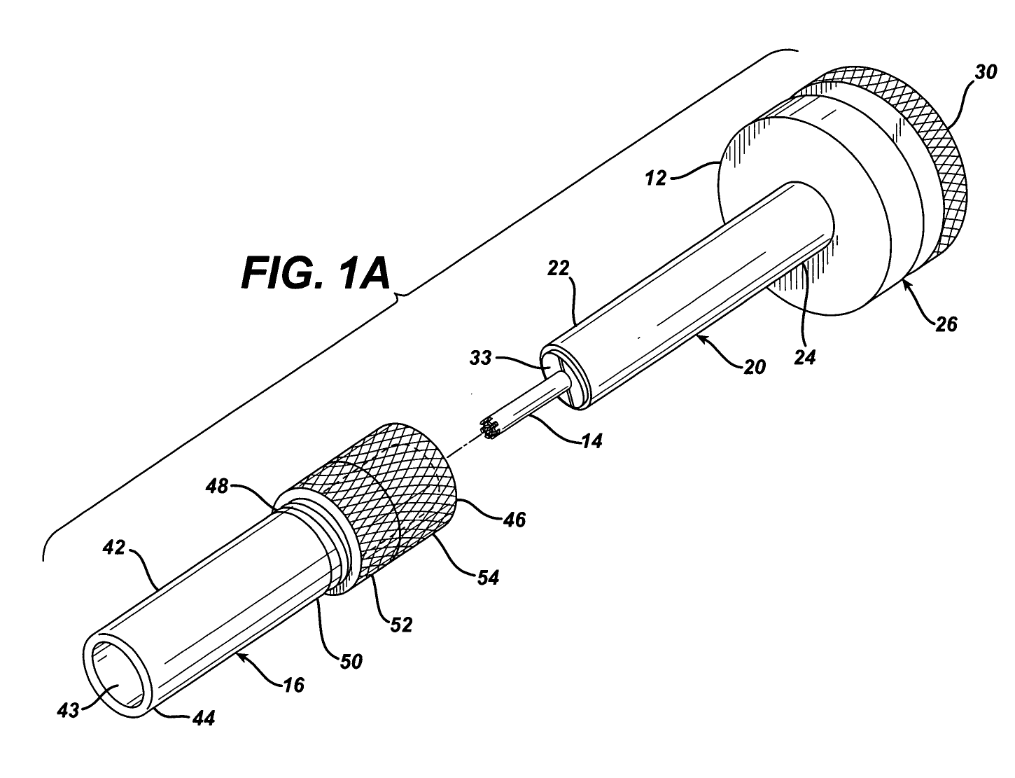

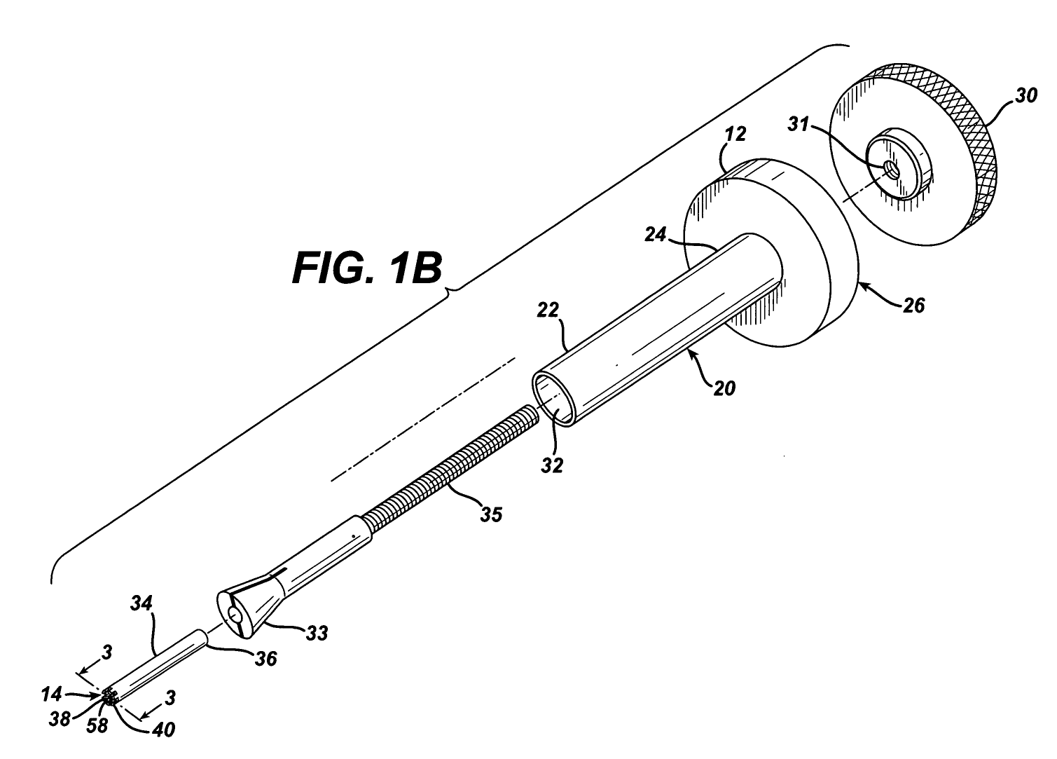

With reference to FIGS. 1 and 1A, in one preferred embodiment, a microactivation device 10 for a skin surface of an individual includes an actuator device 12 having a probe 14 disposed centrally and releasably therein. Optionally, a cylindrical positioning sleeve 16 capable of being secured to actuator device 12 and of being releasably and adjustably positioned over actuator device 12 is provided, so that a desired length 18 of probe 14 may extend therefrom.

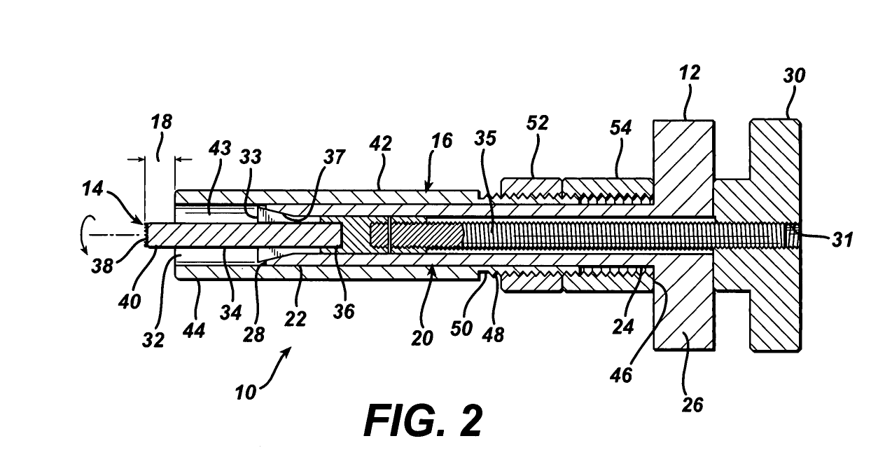

With reference to FIG. 1B, actuator device 12 includes a cylindrical central shaft 20 having a proximal end 22 and a distal end 24 terminating in a handle device 26. Actuator device is further provided with a knurled disk 30 positioned co-axially with shaft 20 and secured substantially centrally onto handle device 26 as will be discussed hereinbelow with reference to FIG. 2. Knurled disk 30 has a threaded opening 31 centrally disposed therein.

With reference to FIG. 2, central shaft 20 has a central opening 32 situated therein, extending from proximal end 22 to distal end 24 thereof, starting with a larger diameter frusta-conical portion 28 at proximal end 22 and forming a seat 37 as the frusta-conical portion tapers into a cylindrical portion and extends toward distal end 24. A collet 33 is releasably seated in seat 37 and releasably fixed to a threaded cylindrical driver rod 35 also disposed within central opening 32, which in turn is connected to knurled disk 30 by threaded screw cooperation at opening 31, thereby securing knurled disk 30 onto handle device 26. Collet 33 is split and compressible so that when it comes into seat 37, it is compressed to hold probe 14.

With reference to FIGS. 1 and 1A, optional positioning sleeve 16 includes a cylindrical rod 42 with a central opening therein 43 of a larger diameter than central shaft 20. Positioning sleeve 16 has a proximal end 44 and a distal end 46, having an outer threaded surface portion 48 extending from a selected position 50 on cylindrical rod 42 and terminating at distal end 46. Positioning sleeve 16 further includes a first locking ring 52 having a threaded cylindrical cavity therein, to cooperate with, and adjustably positioned along, threaded surface portion 48, and a second locking ring 54 having a threaded cylindrical cavity therein to cooperate with threaded surface portion 48 and adjustably positioned along threaded surface 48 from distal end 46 to adjacent first locking ring 52.

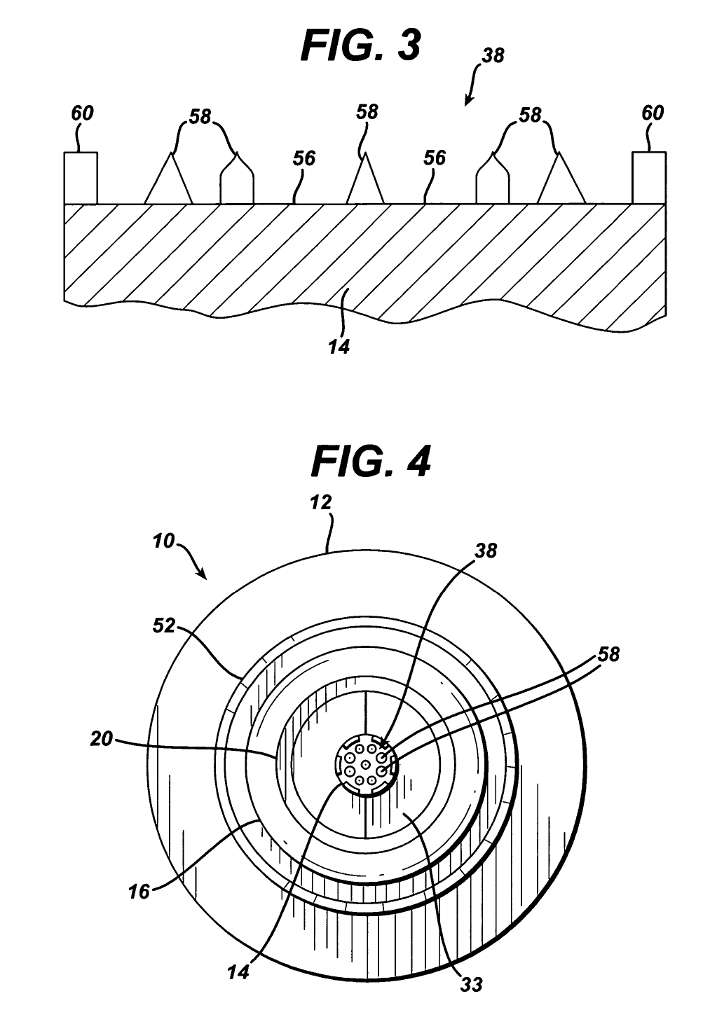

With reference to FIGS. 1B and 3, probe 14 includes a cylindrical rod 34 having a distal end 36 and a proximal end 40 terminating in cross-sectional surface 38, which may be in the form of a disk, and having at least one needle 58 protruding perpendicularly therefrom. Rod 34 may be of any small size suitable for holding one or more needles 58 and appropriately sized to cooperate with collet 33. Preferably, rod 34 is about 1 mm to about 10 mm in cross-sectional diameter. Cylindrical rod 34, and in turn surface 38, preferably has a cross-sectional diameter of about 1.5 mm.

With reference to FIG. 3, surface 38 of probe 14 includes a flat surface area 56 having at least one needle 58 extending upward therefrom and positioned along flat surface area 56 in any configuration, including random or patterned configuration. Preferably, as shown in FIG. 4, the configuration of needles 58 will be patterned, or symmetrical, due to ease of manufacture.

Optionally, according to a preferred embodiment of the present invention, each needle 58 or a plurality of needles 58 is provided with a positioning collar 60 (not shown) extending upward from surface area 56 and surrounding one or more needles 58.

Needle(s) 58 may be of any three-dimensional shape, thereby having any cross-sectional shape and symmetry along either the vertical or horizontal axis, as long as each needle 58 terminates in a sharp point or tip. Preferably, for better activation, the vertical cross-sectional shape of needle(s) 58 is a triangle, cone, frusta-cone, a petal shape or rounded triangle (i.e. a triangle bounded by one straight line and two rounded out lines which meet at 3 points, with the joinder of the two rounded lines forming the sharp point), or another geometry including a sharp tip or angle at the top.

In one aspect of the invention, surface 38 may be comprised of only one needle 58. In another aspect, surface 38 may be comprised of a plurality or array of needles 58. The number and size of needles 58 is determined by the criteria that the level of penetration of needle(s) be no deeper than the epidermis, i.e., just enough to activate the skin but so as not to cause pain. The length of the needles 58 is preferably about 50 micro-m to about 250 micro-m and the depth of penetration is preferably about 100 micro-m. The diameter of the needle(s) at the base can be about 10 micro-m to about 200 micro-m, preferably, about 15 micro-m to about 150 micro-m, and more preferably, about 15 micro-m to about 120 micro-m for most efficient activation. The sharp points or tips of needles 58 have a diameter of about 1 micro-m to about 2 micro-m. Needles 58 may be made of any suitable material for skin micro-penetration, preferably, metal (e.g. stainless steel) or SiO₂, while the rest of device 10 is preferably made of plastic, although it, too, can be made of any suitable material for holding needle(s) 58 and allowing them to make micro-holes in the stratum corneum. A plurality of needles 58 may be arranged as an array on a transdermal patch, such an adhesive patch or a roller, for example, which may be painlessly applied to the epidermal skin, without penetrating the dermis, and thereby microactivate the skin.

With reference to FIGS. 1A and 1B, to assemble microactivation device 10, collet 33 is secured to driver rod 35 and probe 14 inserted into collet 33. Driver rod 35, in turn, is inserted in central opening 32 of central shaft 20 of actuator device 12. Tightening knurled disc 30 over actuator device 12 secures driver rod 35 therein and forces collet 33 into seat 37, thereby gripping probe 14 within collet 33. Positioning sleeve 16 may optionally be slid over central shaft 20, allowing desired length 18 of probe 14 to protrude and extend beyond proximal end 44 of positioning sleeve 16 while second locking ring 54 is at distal end 24 of central shaft 20 of actuator device 12. Desired length 18 is determined by the degree of skin penetration desired in such a way as to substantially avoid penetration of the skin layers below the epidermis. Preferably, the depth of skin penetration is about 100 micro-m.

In use, device 10 is delivered in sterile condition. After sterilizing probe 14 and assembling microactivation device 10, multiple or repetitious strikes against the skin surface are effected manually or by motorized means, by applying microneedle(s) 58 to skin, or contacting the skin surface with needles 58 of probe 14. The angle at which microneedle(s) 58 strike the skin surface may be about 20 deg to about 160 deg., preferably about 70 deg to about 110 deg, and more preferably about 90 deg or perpendicularly relative to the skin surface, as long as the skin is penetrated to the appropriate depth. The number of strikes may be up to about 300 times, preferably about 20 to about 50 times. Generally, skin is penetrated to a depth of about 100 micro-m, causing activation of skin repair functions. Without wishing to be bound by theory, it is believed that the activation of skin repair functions is effected by disruption of the basal layer skin cells.

Further with reference to the use of device 10, second locking ring 54 may be adjusted along threaded surface portion 48, causing it to move closer or further from first locking ring 52, thereby adjusting the length of positioning sleeve 16. Adjusting the overall length of positioning sleeve 16 in turn adjusts the desired length 18 of protrusion of probe 14, thereby allowing for control of pressure applied to the skin and preventing excessive penetration of the skin. Flat surface area 56 of surface 38 of probe 14 also provides control of the depth of needle 58 penetration, as probe 14 will tend not to penetrate beyond the contact of skin with flat surface area 56. Optionally, and preferably, positioning collar(s) 60 also serve the function of control of depth of penetration by adjusting needle 58 extension, such as for different thicknesses of epidermis, as well as to prevent the skin from “bunching up” in the center of probe 14. Preferably, up to about 2% of the skin cells contacted are damaged. After the skin microactivation process is complete, probe 14 is released from collet 33. Probe 14 may optionally be discarded after use.

Microactivation device 10 allows quick, painless skin activation and leaves no residual scar. The cosmetic skin benefits achieved using the inventive microactivation method include:

Fine line and wrinkle reduction;

skin smoothening;

texture improvement, resulting in smooth supple skin with high elasticity;

photodamaged skin repair; and

overall healthier skin appearance, with glow and radiance. The inventive device and method advantageously provide efficiency, minimal discomfort, avoidance of scarring, and the capability to microactivate skin. The device therefore lends itself to multiple use on one individual.

This technique may be capable of adaptation for home-use skin microactivation kits, which may include microactivation device 10 together with instructions for use alone or in combination with skin benefit agents.

Skin Benefit Agents

Retinoids

The inventive compositions contain, as a preferred ingredient, a retinoid, which is selected from retinyl esters, retinol, retinal and retinoic acid, preferably retinol or retinyl ester. The term “retinol” includes the following isomers of retinol: all-trans-retinol, 13-cis-retinol, 11-cis-retinol, 9-cis-retinol, 3,4-didehydro-retinol, 3,4-didehydro-13-cis-retinol; 3,4-didehydro-11-cis-retinol; 3,4-didehydro-9-cis-retinol. Preferred isomers are all-trans-retinol, 13-cis-retinol, 3,4-didehydro-retinol, 9-cis-retinol. Most preferred is all-trans-retinol, due to its wide commercial availability.

Retinyl ester is an ester of retinol. The term “retinol” has been defined above. Retinyl esters suitable for use in the present invention are C₁-C₃₀ esters of retinol, preferably C₂-C₂₀ esters, and most preferably C₂, C₃, and C₁₆ esters because they are more commonly available. Examples of retinyl esters include but are not limited to: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, retinyl oleate.

The preferred ester for use in the present invention is selected from retinyl palmitate, retinyl acetate and retinyl propionate, because these are the most commercially available and therefore the cheapest. Retinyl linoleate and retinyl oleate are also preferred due to its efficacy.

Retinol or retinyl ester is employed in the inventive composition in an amount of from about 0.001% to about 10%, preferably in an amount of from about 0.01% to about 1%, most preferably in an amount of from about 0.01% to about 0.5%.

Essential Fatty Acids. Another preferred optional ingredient is selected from essential fatty acids (EFAs), i.e., those fatty acids which are essential for the plasma membrane formation of all cells. Supplementation of EFA corrects this. EFAs also enhance lipid biosynthesis of epidermis and provide lipids for the barrier formation of the epidermis. The essential fatty acids are preferably chosen from linoleic acid, γ-linolenic acid, homo-γ-linolenic acid, columbinic acid, eicosa-(n-6,9,13)-trienoic acid, arachidonic acid, γ-linolenic acid, timnodonic acid, hexaenoic acid and mixtures thereof.

Phytoestrogens. Phytoestrogens include flavonoids such as 8-prenyl naringenin, hops extracts, soy extracts, chick pea extracts, estrogenic flavonoids, genistein, daidzein, glycitin, biochanin A, formononetin and equol and mixtures thereof, acetyl and malonyl esters of genistein and daidzein, and glucosides of genistein and daidzein. It should be noted that the aforementioned list is not exclusive, and may include other phytoestrogens known to persons of ordinary skill in the art. The present compositions contain from about 0.001% to about 10% of at least one phytoestrogen.

PPAR Activators. Suitable PPAR activators are described in U.S. Pat. No. 6,423,325, incorporated by reference herein. Preferable PPAR activators according to the invention are 12-hydroxystearic acid, cis parinaric acid, trans-7-octadecenoic acid, cis 5, 8,11,14,17eicosapentanoic acid, cis-4,7,10,13,16,19 docosahexenoic acid, conjugated linoleic acid (c9,t11), columbinic acid, linolenelaidic acid, ricinolaidic acid, stearidonic acid, 2-hydroxystearic acid, alpha- linolenic acid, arachidonic acid, cis-11,14-eicosadienoic acid, conjugated linoleic (t10,c12), conjugated linoleic acid (t9,t11), conjugated linoleic acid (50:50 mix of c9, t11 and t10 c12), coriander acids, linolelaidic acid, monopetroselinic acid, petroselinic acid, ricinoleic acid, stearolic acid, thuja extract and trans vaccenic acid. Further suitable preferred PPAR a activators include cis-11,14,17 eicosatrienoic acid, cis-5 eicosenoic acid, cis-8,11,14 eicosatrienoic acid, hexadecatrienoic acid, palmitoleic acid, petroselaidic acid, trans trans farnesol, cis 13, 16 docosadienoic acid, cis vaccenic acid, cis-11 eicosenoic acid, cis-13,16,19 docosatrienoic acid, cis-13-octadecenoic acid, cis-15-octadecanoic acid, cis-7,10,13,16 docosatetraenoic acid, elaidic acid, gamma-linolenic acid, geranic acid, geranyl geranoic acid, linoleic acid, oleic acid, petroselinyl alcohol, phytanic acid, pinolenic acid, trans-13-octadecenoic acid, tridecyl salicylic acid (TDS).

Acidic Skin Benefit Agents. Examples of acidic skin benefit agents include alpha-hydroxy acids and esters, beta-hydroxy acids and esters, polyhydroxy acids and esters, and mixtures thereof. Examples of preferred alpha-hydroxy acids include glycolic acid, lactic acid, 2-hydroxyoctanoic acid, and mixtures thereof. Preferred beta-hydroxy acids include salicylic acid. Examples of preferred dicarboxylic or dioic acid are sebacic acid, malonic acid, and mixtures thereof. Betulinic acid is another example of skin benefit agent that may be used alone or in combination with minoxidil. The amount of the acidic skin benefit agent is at least about 0.001% by weight of the composition.

Skin Lightening Agents. Examples of skin lightening agents include resorcinol derivatives, ferulic acid, kojic acid and esters, hydroquinone, t-butyl hydroquinone, niacinamide, Vitamin C derivatives, and mixtures thereof. Specifically, certain resorcinol derivatives, particularly 4-substituted resorcinol derivatives, are useful in cosmetic compositions for hair and skin benefits among others. Resorcinol derivatives are described in many publications, including Hu et al., U.S. Pat. No. 6,132,740 and European Patent Application EP 1 134 207. Preferred are 4-alkyl resorcinols, such as 4-ethyl resorcinol. Other effective skin lightening resorcinol derivatives include 4,6-di-substituted resorcinol derivatives, such as 4,6-di-alkyl resorcinols.

Sebum Control Agents. Another skin benefit is sebum suppression. Examples of sebum control agents include carboxyalkylates of branched alcohols and ethoxylates thereof as disclosed, for example, in WO 99/18928. Applicants' co-pending U.S. patent applications Ser. No. 09/872,897, filed Jun. 1, 2001; Ser. No. 10/196,881 filed Jul. 17, 2002; and Ser. No. 10/196,770 filed Jul. 17, 2002, disclose cosmetic methods and compositions for conditioning human skin by topical application to the skin of cosmetic compositions containing carboxyalkylates of branched alcohols, and/or ethoxylates thereof.

Wound Healing Agents. It is particularly beneficial to use microactivation device 10 in combination with wound healing agents, including: zinc compounds such as zinc oxide and zinc gluconate; live yeast cell derivatives, aloe vera extract and compounds derived from aloe vera plant; lipoteichoic acid; Centella asiatica extracts; asiaticosides; eupolin, copper peptides; growth factors such as TGF b, PDGF; natural extracts such as witch hazel, chamomile, calendula and others having wound healing properties.

Astringents. Examples of astringents include, but are not limited to, ethanol, witch hazel, zinc and aluminum salts, and polyphenols. Aluminum chlorohydrate, referred to herein in shortened form as ACH, is the most preferred astringent salt for the purposes of the present invention, due to its wide commercial availability and relatively low cost.

Other skin benefit agents may be included, such as vitamins A, B1, B2, B6, D, E, dexpanthenol and beta carotene, coenzyme Q10, carnitine, and peptides consisting of 3 to about 100 amino acids, as well as electrical, magnetic, or electromagnetic stimulation of skin. The said invention is also beneficial when used in combination with actives having a logP of greater than or equal to 5, molecular weights of greater than or equal to about 400 Daltons, or combinations thereof.

Additional cosmetic skin benefit agents include liquid, inert, hydrophobic fluorocarbon infused with carbon dioxide, as descibed in U.S. Pat. No. 5,851,544, incorporated by reference herein. The compositions increase blood flow to the skin thus increasing endogenous oxygen and nutrient delivery to the skin. Such cosmetic skin care compositions may include: i) from about 0.1% to about 70%, by weight of the composition, of a fluorocarbon infused with carbon dioxide wherein the fluorocarbon is inert, liquid at 25.degree. C. and hydrophobic and is selected from the group consisting of perfluorooctane, perfluorodecane, perfluorodecalin, perfluorooctylbromide, perfluorodecylbromide, perfluorooctyliodide, perfluorotripropylamine, perfluoro-tributylamine, bis-(F-butyl)-ethene and perfluoro-polymethylisopropyl ether. The oxygen will provide the energy to enhance cell proliferation during the healing of the microwound caused by the microactivation device.

Optional Components

Sunscreens. Sunscreens include those materials commonly employed to block ultraviolet light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively.

The exact amount of sunscreen employed in the emulsions can vary depending upon the degree of protection desired from the sun's UV radiation.

Other optional ingredients may include coloring agents, opacifiers and pigments (e.g. titanium dioxide, silica) and perfumes. Amounts of these materials may range anywhere from 0.001% up to 20% by weight of the composition.

Many cosmetic compositions, especially those containing water, must be protected against the growth of potentially harmful microorganisms. Anti-microbial compounds, such as triclosan, and preservatives are, therefore, necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Cosmetically Acceptable Vehicle. The compositions according to the invention also comprise a cosmetically acceptable vehicle to act as a dilutant, dispersant or carrier for the skin benefit ingredients in the composition, so as to facilitate their distribution when the composition is applied to the skin.

The vehicle may be aqueous, anhydrous or an emulsion. Preferably, the compositions are aqueous or an emulsion, especially water-in-oil or oil-in-water emulsion. Water when present will be in amounts which may range from 5 to 99%, preferably from 40 to 90%, optimally between 60 and 90% by weight.

Suitable oils and/or emollients include but are not limited to silicone oil, vegetable oils, esters, fatty acids and alcohols, and hydrocarbons. Levels of such oil and/or emollients may range from about 0.5% to about 50%, preferably about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol from the B.F. Goodrich Company. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, Fullers earth, kaolin, starch, smectite clays, chemically modified magnesium aluminum silicate, organically modified montmorillonite clay, hydrated aluminum silicate, fumed silica, aluminum starch octenyl succinate and mixtures thereof.

The device and compositions of the cosmetic system of the present invention are intended primarily as a product for topical application to human skin, especially as an agent for conditioning and smoothening the skin, and preventing or reducing the appearance of wrinkled or aged skin.

In the following, several examples of application of the inventive system and method are described. The following is by way of example, not by way of limitation, of the principles of the invention to illustrate the best mode of carrying out the invention.

### EXAMPLE 1

This example demonstrates the use of the inventive microactivation methods to provide efficient epidermal tissue microactivation.

Cell Culture

Normal human keratinocytes, isolated from neonatal foreskin by trypsin treatment, were grown in Dulbeco's Modified Eagles Medium (DMEM, from Life Technologies, Grand Islands, N.Y.) with 10% fetal bovine serum in the presence of irradiated mouse fibroblasts for establishing dividing keratinocyte colonies. Cells were incubated until their second passage and stored at −70° C. for future use. All incubations took place at 37° C. with 5% CO₂. Frozen second passage keratinocytes were thawed and plated in T-75 flasks (Corning, Corning N.Y.) in KGM medium (Clonetics Corp., San Diego, Calif.), grown to confluence and harvested with trypsin treatment for further use.

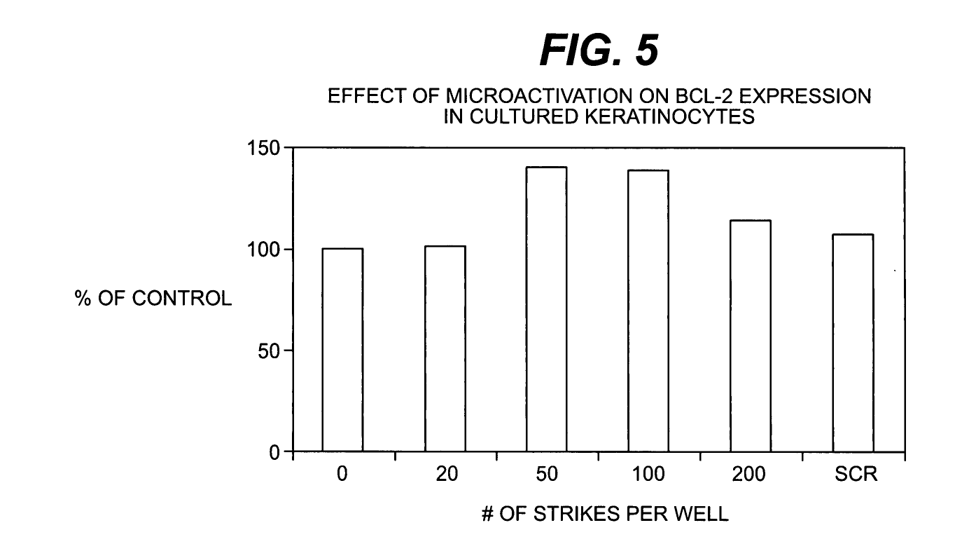

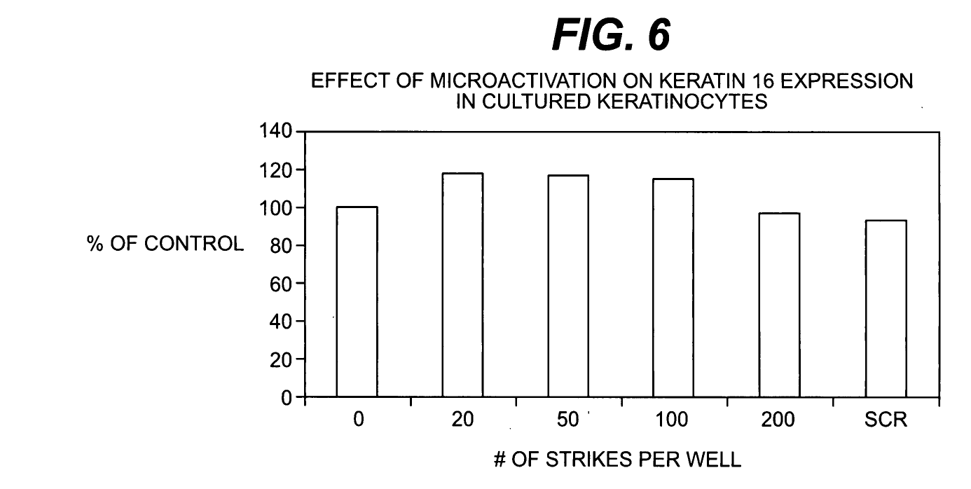

Keratinocytes were plated in six well plates (Corning) at a density of 1 million per plate and grown to confluence. At confluence, medium was replaced with fresh KGM and a sterile aliquot of 0.2 M CaCl₂ solution was added to bring the calcium concentration to 1.2 mM. Cells were incubated for an additional 24 hours. The medium was replaced with fresh KGM. The cells were wounded with needles 58 that were acupuncture needles (Peace brand, CT5P-01, China, 0.16 mm×13 mm) mounted on surface 56, in the following manner. Five sterile needles 58 were held so that the tips aligned; this grouping was pressed into the confluent bed of cells 0, 20 50, 100 and 200 times in a random distribution pattern so that needle 58 strikes were evenly placed across the plate floor. In one well the needles were dragged across the plate floor to achieve one passage of the needle grouping over the entire floor area. This well was referred to as the “scratch” well. Cells were incubated for another 24 hours. The medium was then removed and the cells washed with phosphate buffered saline solution (PBS, 0.85% NaCl, 10 mM NaPO₄, pH 7.4) and lysed with 1 ml of extraction buffer (PBS, 0.5% sodium lauryl sulfate, 4 M urea, 10 mM EDTA). The lysate was used to prepare electrophoresis/Western blotting samples and antibodies for BCL-2 (Santa Cruz Biotechnology, Santa Cruz, Calif.) and keratin 16 (Labvision, Fremont Calif.) were used to quantify those antigens by using the Typhoon imaging system (Pharmacia). Results were expressed as percent of control and are shown in FIGS. 5 and 6.

Results showed that both BCL-2 and keratin 16 were elevated in the wells with wounded keratinocytes in a dose-dependent manner. Both BCL-2 and keratin 16 are regarded as markers for keratinocyte “activation”, a state in which they are engaged in the process of wound healing.

### EXAMPLE 2

Normal human fibroblasts, isolated from neonatal foreskin by trypsin treatment, were grown in Dulbeco's Modified Eagles Medium (DMEM, from Life Technologies, Grand Islands, N.Y.) with 10% fetal bovine serum. Cells were incubated until their second passage and stored at −70° C. for future use. All incubations took place at 37° C. with 5% CO₂. Frozen second passage fibroblasts were thawed and plated in T-75 flasks (Corning, Corning N.Y.) in DMEM medium, grown to confluence and harvested with trypsin treatment for further use.

Fibroblasts were plated at a density of 1 million cells per twelve well plate and grown to confluence with the incubation conditions described above. Keratinocytes cultured as described above were wounded as follows. A group of five acupuncture needles 58 (as described above in Example 1) was dragged across the floor of the wells 0, 5, 10 or 20 times in an evenly distributed pattern. After wounding, cells were incubated for 24 hours. The 2 ml of medium from each well were then harvested and 1 ml was placed in one of the wells of a 12 well plate containing fibroblasts along with 1 ml of fresh DMEM. Samples were produced in triplicate. Plates were then incubated for an additional 8, 24 or 48 hours. The supernatant medium was then harvested and aliquots were used for electrophoresis and Western blotting to determine the presence and quantity of procollagen 1 with the appropriate antibody (Chemicon, Temecula, Calif.).

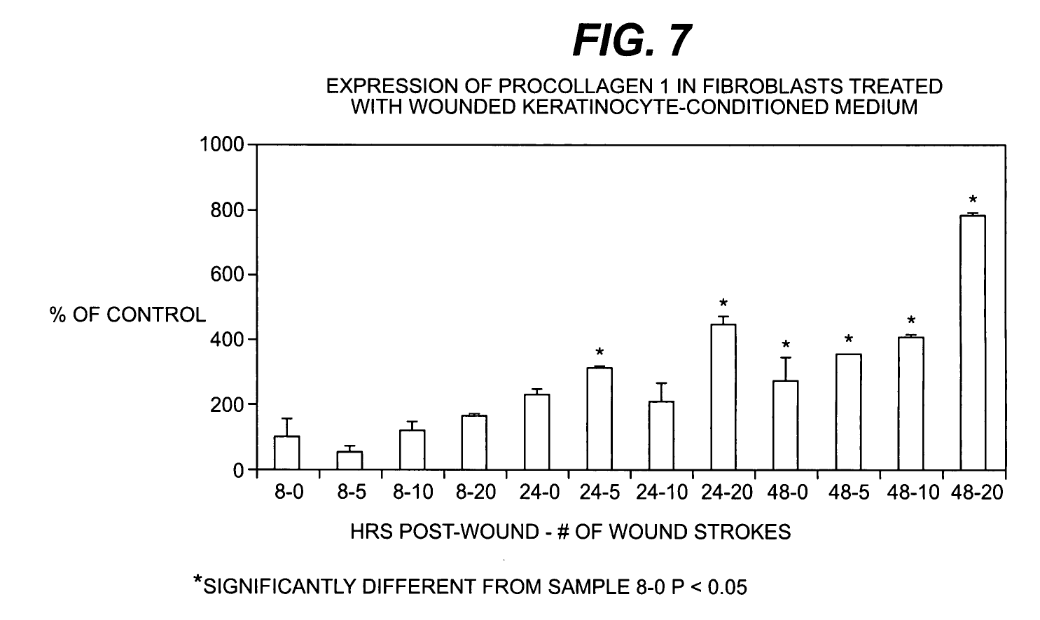

Procollagen 1 is a precursor of collagen and is considered to play an essential role in wound healing and dermal regeneration. With reference to FIG. 7, the results showed that the supernatant medium from wounded keratinocytes harvested 8, 24 and 48 hours after wounding, was able to elevate procollagen 1 expression in cultured fibroblasts.

### EXAMPLE 3

### Clinical Trials

Ten subjects between the ages of 25 and 50 were recruited into a clinical trial to test the efficacy of microneedles 58 after having given informed consent to a protocol approved by an internal review board. Needles 58 that are acupuncture needles (Seirin, 0.12 mm×30 mm) were fixed in narrow diameter plastic tubes, i.e., positioning collars 60, with adhesive so that needle tips protruded approximately 250 microns beyond the end of positioning collar 60. This arrangement prevented excessive penetration during use. The protocol provided for the application on the skin of the lower back of a vehicle alone or vehicle containing either 0.05% or 0.1% retinol. In vehicle alone sites and 0.05% retinol sites microneedles 58 were applied at a density of 100 strikes per square centimeter. Adhesive rings with an internal area of 1.5 square centimeters were used to define the treatment areas. Microneedle device 10 was held in the operator's hand and lightly impacted on the subject's skin. Aliquots of twenty microliters of treatment solution were applied to the relevant sites immediately after microneedling. Sites were treated once a day for four days. On the fifth day epidermal samples were taken with an abrasive probe and placed in extraction buffer (see above). Samples were analysed with polyacrylamide gel electrophoresis and Western blotting for the presence of cellular retinoic acid binding protein II (CRABP II), a protein considered to be a marker for retinoid activity in skin.

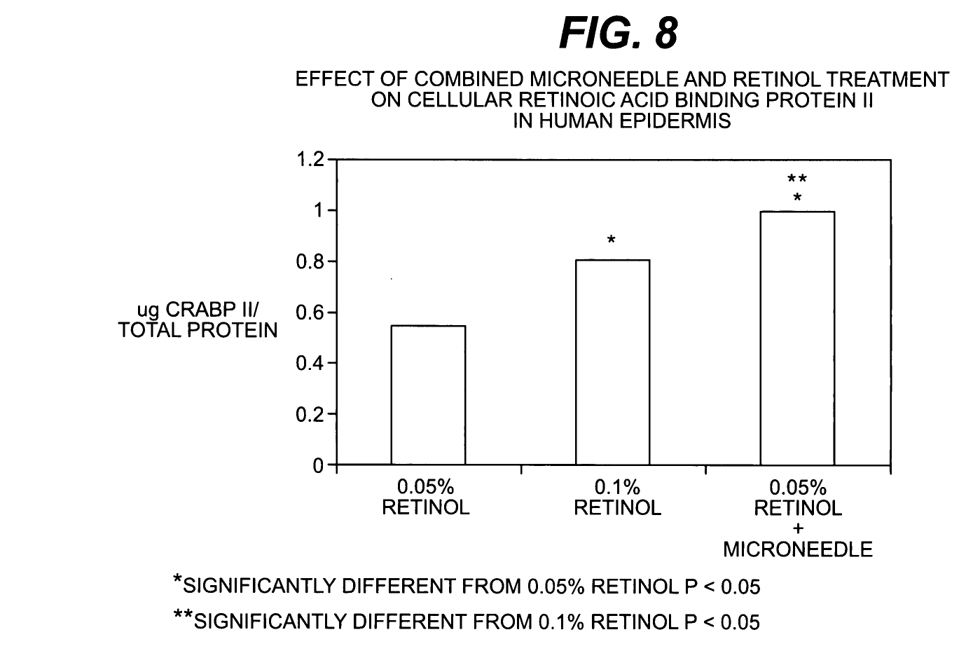

With reference to FIG. 8, results showed a dose-dependent increase in CRABP II with increasing concentration of applied retinol. Results also showed that microneedle 58+0.05% retinol treatment increased CRABP II expression above that seen with 0.05% retinol alone in a statistically significant manner (P<0.05) and, in fact, that the CRABP II expression elevation associated with microneedles 58+0.05% retinol treatment was greater than that associated with the 0.1% retinol alone treatment. This difference was also statistically significant.

### EXAMPLE 4

### Microneedle Induction of BCL-2 Expression

This trial was intended to test the efficacy of microneedle 58 treatment alone in elevating a biochemical in skin considered to be a marker for the activation (transition to a wound healing state) of skin cells. An increase in expression of BCL-2 biomaterial after cellular wounding is considered to be a marker for activation.

Ten subjects between the ages of 25 and 50 were recruited into a clinical trial to test the efficacy of microneedles 58 after having given informed consent to the approved protocol. Microneedles 58 (as in Example 3 above) were applied at a density of 100 strikes per square centimeter to skin on the subject's back. Adhesive rings with an internal area of 1.5 square centimeters were used to define the treatment areas. One site served as an untreated control; nine other sites were given microneedle 58 treatment once a day for one, three and nine days. Epidermal abrasion samples were taken on the first, third and tenth day and processed as described above. At day one, samples were taken from the untreated control site and from three separate treated sites 15 minutes, 5 hours and 24 hours after treatment. On day three samples were taken from one site previously treated but not treated on day three and three samples from three separate sites 15 minutes, 5 hours and 24 hours after treatment. On day ten two sites were sampled, one which had been previously treated but was not treated on day ten and another 15 minutes after treatment. Samples were analysed with polyacrylamide gel electrophoresis and Western blotting for the presence of a protein called BCL-2.

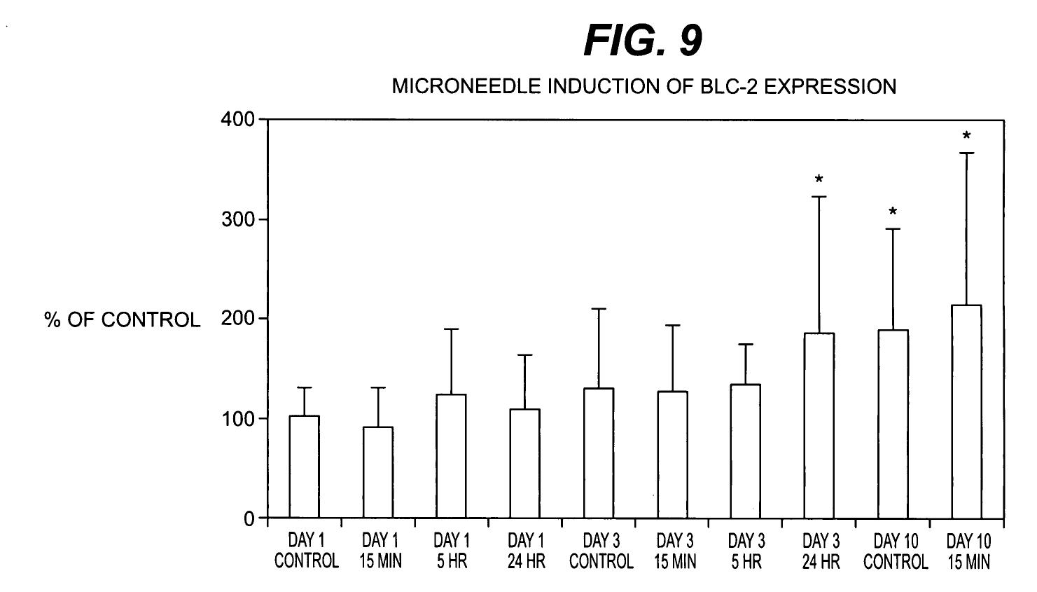

With reference to FIG. 9, results showed a statistically significant increase in the level of BCL-2 in samples taken from subjects 24 hours after day three treatment and on both day ten samples. This indicates that microneedle 58 treatment induced activation in epidermal cells. Clinicians did not observe any significant irritation at any treated site. None of the microactivation sites exhibited any scarring from the epidermal microactivation procedure.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable. Throughout this application, various publications have been cited. The entireties of each of these publications are hereby incorporated by reference herein.

1. A microactivation system comprising:
(a) a microactivation device for microactivation of a skin surface of an individual, comprising:
an actuator device comprising a central shaft having a proximal end and a distal end conjoined with a handle device; said central shaft having a central opening therein extending from said proximal end to said distal end;
a probe capable of being disposed centrally and releasably within said central opening of said actuator device; said probe comprising a cylindrical rod having a distal end and terminating in a surface at a cross-sectional proximal end thereof; said surface having a small cross-sectional diameter and having at least one needle protruding therefrom; and
(b) A skin benefit agent.

2. The system of claim 1, wherein said actuator device further comprises:
a knurled disc positioned co-axially with said shaft, said knurled disc having a threaded opening centrally disposed therein; and
a threaded cylindrical driver rod in screw cooperation with said knurled disc; and
wherein said system further comprises a collet seated within said central opening and releasably fixed to said cylindrical driver rod.

3. The system of claim 1, wherein said surface of said probe includes a plurality of needles extending therefrom; and wherein said surface is selected from a group consisting of adhesive patch, roller device, and combinations thereof.

4. The system of claim 1, wherein said needles have a cross-sectional geometric shape that includes a triangle, trapezoid, or a combination thereof.

5. The system of claim 1, wherein said needles have a geometry of a frustacone.

6. The system of claim 1, wherein said skin benefit agent is selected from the group consisting of retinoids, essential fatty acids, PPAR activators, phytoestrogens, acidic skin benefit agents, skin lightening agents, sebum control agents, astringents, and combinations thereof.

7. The system of claim 1, wherein said at least one needle protrudes about 250 micro-m beyond said surface of said probe.

8. A method for microactivation of epidermal cells of a skin surface of an individual, comprising providing the microactivation system of claim 1; wherein upon being applied to said skin surface with said surface, said needles microactivate said epidermal tissue.

9. The method of claim 8, wherein said skin benefit agent is applied to the skin about 1 minute to about 24 hours preceding or following said application of said needles.

10. The method of claim 8, wherein after said microactivation is complete, said probe is released from said actuator device.

11. The method of claim 8, wherein said skin benefit agent is selected from the group consisting of retinoids, essential fatty acids, PPAR actives, phytoestrogens, acidic skin benefit agents, skin lightening agents, sebum control agents, wound healing agents, astringents, and combinations thereof.

12. The method of claim 8, wherein said microneedle(s) are applied relative to said skin surface at an angle of about 20 deg to about 160 deg.

13. The method of claim 8, wherein said microactivation provides a skin benefit selected from the group consisting of smoothening of the skin, reducing the appearance of fine lines and wrinkles and sagging skin, improved texture of smooth supple skin with high elasticity, repair of photodamaged skin, firmness, glow, overall healthy skin appearance, and combinations thereof.

14. A kit including the system of claim 1.

15. The kit of claim 14, wherein use of said kit by a consumer does not require the assistance of a physician.

16. A method for assembling the microactivation device of the system of claim 3, comprising:
(a) securing said collet to said driver rod,
(b) inserting said probe into said collet,
(c) inserting said secured driver rod in said central opening of said central shaft of said actuator device; and
(d) tightening said knurled disc over said actuator device, thereby securing said driver rod therein and forcing said collet to grip said probe.

17. The method for assembling the microactivation device according to claim 16, wherein said device is applied to the skin in such a way as to substantially avoid penetration of the skin layers below the epidermis.

18. The method for assembling the microactivation device according to claim 17 wherein said device enables skin penetration is to a depth of about 100 micro-m.

19. A method of skin microactivation comprising applying to the skin a device selected from the group consisting of
(a) a microactivation device for microactivation of a skin surface of an individual, comprising:
an actuator device comprising a central shaft having a proximal end and a distal end conjoined with a handle device; said central shaft having a central opening therein extending from said proximal end to said distal end; a probe capable of being disposed centrally and releasably within said central opening of said actuator device; said probe comprising a cylindrical rod having a distal end and terminating in a surface at a cross-sectional proximal end thereof; said surface having a small cross-sectional diameter and having at least one needle protruding therefrom;
(b) a device substantially as described in any of U.S. Pat. Nos. 5,879,326 and 5,591,139; and
(c) combinations and substantial equivalents thereof.

20. The method according to claim 19, wherein said application of said device to said skin comprises repeated applications of said device to the skin.
